(19) United States
(12) Patent Application Publication
(10) Pub. No.: US 2009/0318768 A1
(43) Pub. Date: Dec. 24, 2009

(54) Laryngoscope and Method of Use

(76) Inventors: James P. Tenger, Carlsbad, CA (US); Leslie A. Tenger, Carlsbad, CA (US); John R. Hicks, Carlsbad, CA (US)

Correspondence Address: PROCOPIO, CORY, HARGREAVES & SAVITCH LLP, 530 B STREET, SUITE 2100, SAN DIEGO, CA, 92101, US

(21) Appl. No.: 12/368,952

(22) Filed: Feb. 10, 2009

Related U.S. Application Data
(63) Continuation-in-part of application No. 12/173,961, filed on Jul. 16, 2008.
Application No. 12/173,961 is a continuation-in-part of application No. 12/144,147, filed on Jun. 23, 2008.

(51) Int. Cl. A61B 1/267

(52) U.S. Cl. 600/194

(57) ABSTRACT

A laryngoscope for use in viewing the vocal cords of a patient in performance of an endotracheal intubation includes a handle and a laryngoscope blade connected to the upper end of the handle to lift the patient's tongue and mandible for viewing the vocal cords and aid in the insertion of an endotracheal tube. The handle is bent or curved along its length with the lower end portion bent or curved inwardly towards the blade, and finger gripping indents are formed on the inner surface of the handle, along with a blade stop adjacent the handle to restrict contact between the blade and handle. A black light source may be carried by the blade.

### CROSS-REFERENCE TO RELATED APPLICATIONS

This application is a continuation-in-part of U.S. patent application Ser. No. 12/173,961 filed on Jul. 16, 2008, which is a continuation-in-part of U.S. patent application Ser. No. 12/144,147, filed on Jun. 23, 2008, the contents of each of which are incorporated herein by reference in their entirety.

### FIELD OF THE INVENTION

The field of this invention relates to laryngoscopes and the laryngoscopy procedure.

### BACKGROUND OF THE INVENTION

Health care providers perform direct laryngoscopy to either clear a patient's airway of debris, or place an endotracheal tube into a patient's trachea to assist or replace a patient's ability to oxygenate his/her system. The laryngoscope utilizes either a lighted straight or curved blade that allows visualization of the vocal cords, indicating the opening of the trachea. This lighted blade is used to keep the tongue from obstructing the medical provider's view of the vocal cords. The tip of the blade lifts the epiglottis, thereby providing a direct view into the patient's trachea, and reducing the risk of intubating the esophagus instead. Laryngoscopes in the past have used halogen or LED bulbs to provide a white light source on the laryngoscope blade to illuminate the vocal cords during laryngoscopy. Even with a light source on the laryngoscope blade, viewing of the vocal cords has still proven difficult during laryngoscopy. Accordingly, without the ability to view the vocal cords, many possible laryngoscopy procedures are not performed successfully, resulting in additional harm or death for the patient.

### SUMMARY OF THE INVENTION

To solve these problems and others, an aspect of present invention involves a laryngoscope including an improved light source. The laryngoscope blade carries a black light source that emits a black light during laryngoscopy. The black light enhances visualization of the vocal cords so that visualization of the endotracheal tube passing through the vocal cords is enhanced. The black light emitted from the laryngoscope enhances colors that are white and/or contain phosphors in the region of the vocal cords, enhancing visualization of the target vocal cords. In another implementation of the laryngoscope, the laryngoscope blade carries a black light source and a white light source. A switch on the laryngoscope enables the health care provide to actuate the black light source, the white light source, or to activate both light sources.

An additional aspect of the invention involves a laryngoscope for use in viewing the vocal cords of a patient in performance of an endotracheal intubation. The laryngoscope includes a handle to be gripped by a medical professional in performing the endotracheal intubation; a blade portion extending from the handle to lift the patient's tongue and mandible for viewing the vocal cords and aid in the insertion of an endotracheal tube; a power source; and a black light source powered by the power source and carried by the blade portion to prompt the visible effects of fluorescence and phosphorescence with respect to the patient's vocal cords for viewing the vocal cords and passing of the endotracheal tube there between during endotracheal intubation.

Another aspect of the invention involves an endotracheal intubation system for performing an endotracheal intubation including the laryngoscope described immediately above; and an endotracheal tube insertable into the patient's mouth, between the patient's vocal cords into the patient's larynx, and into the patient's trachea for opening the patient's airway.

A further aspect of the invention involves a method of performing an endotracheal intubation including using the laryngoscope described immediately above to lift the patient's tongue and mandible for viewing the vocal cords and aid in the insertion of an endotracheal tube; illuminating the patient's vocal cords with the black light source to prompt the visible effects of fluorescence and phosphorescence with respect to the patient's vocal cords for viewing the vocal cords; and simultaneously visualizing the patient's vocal cords with the black light source and sliding the endotracheal tube between the patient's vocal cords and into the patient's larynx and trachea.

A still further aspect of the invention involves a laryngoscope blade for use in viewing the vocal cords of a patient in performance of an endotracheal intubation. The laryngoscope blade includes a laryngoscope blade portion connectable to a laryngoscope handle to lift the patient's tongue and mandible for viewing the vocal cords and aid in the insertion of an endotracheal tube; and a black light source carried by the blade portion to prompt the visible effects of fluorescence and phosphorescence with respect to the patient's vocal cords for viewing the vocal cords and passing of the endotracheal tube there between during endotracheal intubation.

According to another embodiment, a laryngoscope for use in viewing the vocal cords of a patient in performance of an endotracheal intubation includes a handle to be gripped by a medical professional in performing the endotracheal intubation, and a blade portion pivotally connected to an upper end of the handle and extending from the handle to lift the patient's tongue and mandible for viewing the vocal cords and for aiding in the insertion of an endotracheal tube. The handle is of an ergonomically curved or angled overall shape, and has an inner surface with a series of arcuate finger grip indents. In one embodiment, an outwardly projecting blade stop is formed integrally on the inner surface of the handle adjacent the upper end, and prevents the blade from touching the handle. The handle may be curved continuously along all or most of its length in an ergonomic shape for comfortable gripping by the user. Alternatively, the handle may have a first portion extending from one end up to an angled bend, and a second portion extending from the bend at an angle to the first portion.

Further objects and advantages will be apparent to those skilled in the art after a review of the drawings and the detailed description of the preferred embodiments set forth below.

### BRIEF DESCRIPTION OF THE DRAWINGS

FIG. 1 is a schematic depiction of a laryngoscope being used to view a patient's larynx;

FIG. 2 is a perspective view of a laryngoscope in accordance with an embodiment of the present invention;

FIG. 3A is a bottom plan view of the laryngoscope of FIG. 2, and illustrates an embodiment of a switch that may be used with the of the laryngoscope of FIG. 2;

FIG. 3B is a bottom plan view of an alternative embodiment of a laryngoscope, and illustrates an embodiment of a black light source on the bottom of the laryngoscope;

FIG. 4 is an embodiment of an endotracheal tube that may be used with the laryngoscope of FIGS. 1-3.

FIG. 5 is an embodiment of a kit of endotracheal tubes that may be used with the laryngoscope of FIGS. 1-3.

FIG. 6 is an alternative embodiment of an endotracheal tube that may be used with the laryngoscope of FIGS. 1-3 or a standard laryngoscope.

FIG. 7 is an embodiment of a pair of protection intubation glasses that may be used with the laryngoscope of FIGS. 1-3 and/or the endotracheal tube(s) of FIGS. 4-6.

FIG. 8 is a side-elevational view of a separate laryngoscope blade in accordance with an embodiment of the present invention;

FIG. 9 is a side-elevational view of a separate laryngoscope blade in accordance with another embodiment of the present invention;

FIG. 10 is a side-elevational view of an embodiment of a pair of forceps;

FIG. 11 is a rear perspective view of an alternative embodiment of a laryngoscope with an ergonomically shaped handle;

FIG. 12 is a side elevation view of the laryngoscope of FIG. 11;

FIG. 13 is a front perspective view of the laryngoscope of FIGS. 11 and 12;

FIG. 14 is a cross-sectional view of the laryngoscope on the lines 14-14 of FIG. 11;

FIG. 15 is a rear perspective view of another embodiment of a laryngoscope with a shaped handle; and

FIG. 16 is a side elevation view of the laryngoscope of FIG. 15.

FIG. 17 is a perspective view of another embodiment of a laryngoscope.

FIG. 18 is a bottom plan view of the laryngoscope illustrated in FIG. 17.

FIG. 19 is a front elevational view of the laryngoscope illustrated in FIG. 17.

FIG. 20 is a right side elevational view of the laryngoscope illustrated in FIG. 17.

FIG. 21 is a left elevational view of the laryngoscope illustrated in FIG. 17.

FIG. 22 is a rear elevational view of the laryngoscope illustrated in FIG. 17.

FIG. 23 is a top plan view of the laryngoscope illustrated in FIG. 17.

### DETAILED DESCRIPTION OF PREFERRED EMBODIMENTS

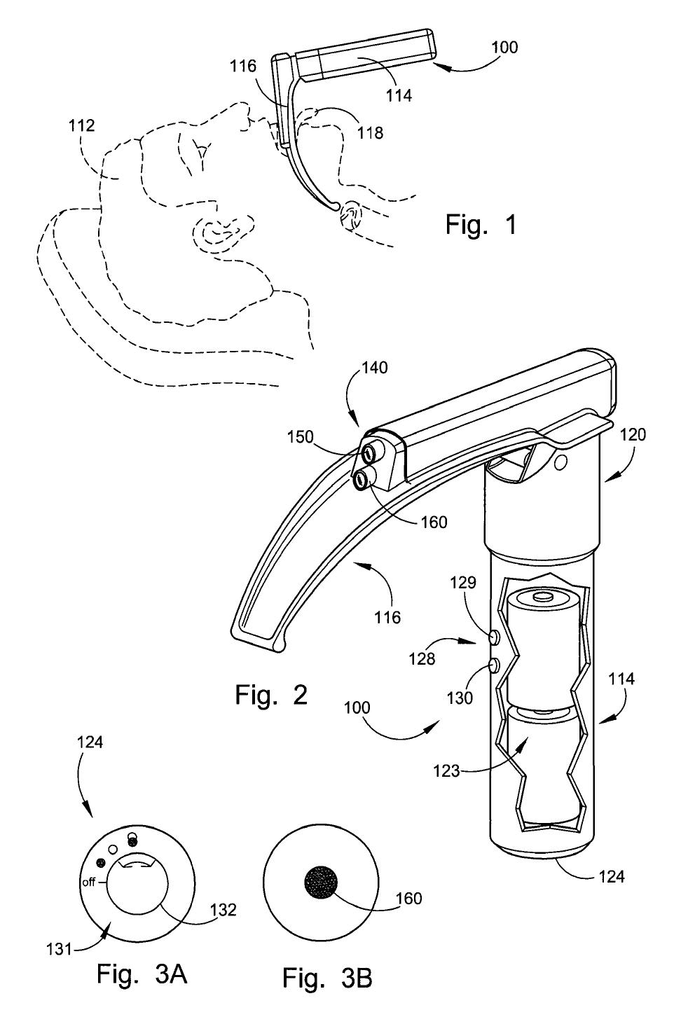

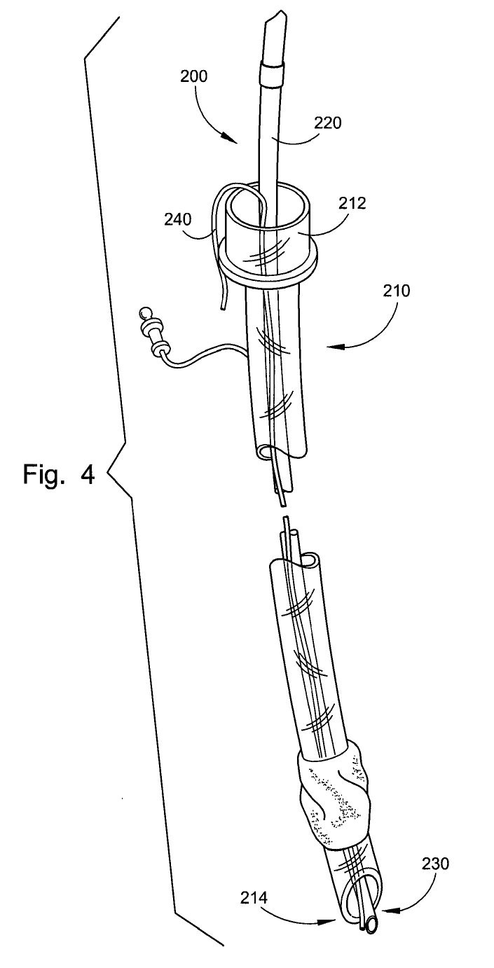

With reference to FIGS. 1 and 2, a laryngoscope 100 constructed in accordance with an embodiment of the invention will be described. In FIG. 1, the laryngoscope 100 is shown placed in the mouth of a patient 112 for viewing the vocal cords adjacent the larynx and to aid in the insertion of an endotracheal tube 200 (FIG. 4) past the vocal cords. The laryngoscope 100 includes a handle or handgrip 114 and a blade portion 116, the latter being used to lift the tongue and mandible 118 out of the way for viewing the vocal cords.

As shown in FIG. 2, the blade portion 116 is pivotally connected to a handle cap 120. The blade portion 116, the handle cap 120, and the handle 114 can be readily assembled together for use. In the embodiment shown, when assembled, a white light source 150 and a black light source 160 are automatically actuated. In an alternative embodiment, the blade portion 116 is directly connected to a top of the handle 114 (i.e., there is no handle cap). The handle 114 is essentially a hollow tube having a closed lower or outer end 122 (FIG. 3) and an open upper end. The handle cap 120 is removably secured to an open upper end of the handle 114. A power source 123 (e.g., one or more dry cell batteries) is located within the handle cavity to provide power for light source(s) to be described.

A side of the handle 114 includes a switch 128 in the form of two pressure/push buttons 129,130 interconnected with the power source 123, the white light source 150, and the black light source 160 that, when pressed (e.g., by a user's thumb), deactivate the respective light sources 150,160. When the laryngoscope 100 is clicked together/assembled, both light sources 150,160 are placed in electrical communication with the power source 123 and are automatically turned on. In the embodiment shown, push button (“white light button”) 129 controls the white light source 150 so that pushing on the white light button 129 turns off the white light source 150 and push button (“black light button”) 130 controls the black light source 160 so that pushing on the black light button 130 turns off the black light source 160. Thus, by applying pressure to the push buttons 129, 130, the respective light sources 150, 160 are turned off. Removing the pressure to the push buttons 129, 130 causes the respective light sources 150, 160 to be turned back on. Accordingly, the switch 128 enables the laryngoscope 100 to be switched between at least a “both on” condition where both black light is emitted from the black light source 160 and white light is emitted from the white light source 150, a black light condition where black light is emitted from the black light source 160 and the white light source 150 is off, and a white light condition where white light is emitted from the white light source 150 and the black light source 160 is off.

With reference to FIG. 3, an alternative embodiment of a switch 131 located on a bottom 124 of the handle 114 is shown. The switch 131 cooperates with electronics in the laryngoscope 100 for switching between an “off” condition, a black light condition, a white light condition, and a “both on” condition. In the embodiment shown, the switch 131 is a rotating switch with a rotating knob 132 that rotates between different positions to actuate the above conditions.

In alternative embodiments, other types of switches may be used (e.g., push-button switch, a toggle switch) on the bottom 124 of the handle 114 (or at other locations on the laryngoscope 100) to switch between an “off” condition and one or more of a black light condition, a white light condition, and a “both on” condition. In alternative embodiments, the switch 128, 131 may switch between conditions in addition to or other than an “off” condition, a black light condition, a white light condition, and a “both on” condition. For example, in an alternative embodiment, where the laryngoscope 100 includes only a black light source 160, the switch 128, 131 may switch the laryngoscope 100 between an “off” condition and a black light condition.

One or more light sources 140 are interconnected with the power source 123 in the handle 114. In the embodiment shown, as discussed above, the one or more light sources 140 include two light sources: 1) a white light source 150, and 2) a black light source 160. Example white light sources 150 include, but not by way of limitation, a white halogen light, a white incandescent light, and a white LED. The black light source 160 emits long wave UVA radiation and very little visible light. The black light source is a lamp emitting electromagnetic radiation that is almost exclusively in the soft near ultraviolet range. The black light source 160 prompts the visible effects of fluorescence and phosphorescence with respect to the patient's vocal cords and the glottis, which is the space between the vocal cords, during laryngoscopy. The black light source 160 may be a Wood's light made using Wood's glass, which is a nickel-oxide-doped glass, which blocks substantially all visible light above 400 nanometers. In alternative embodiments, other black light sources 160 (e.g., LED(s)) may be used. Preferably, the black light source 160 emits electromagnetic radiation including a wavelength in the range of 315 to 400 nm, with out emitting substantial electromagnetic radiation including a wavelength outside of the range of 315 to 400 nm. In a more preferred embodiment, the black light source 160 includes a wavelength of 385-395 nm.

In the embodiment shown, the blade portion 116 is a curved Macintosh blade; however, in alternative embodiments, other types of blades (e.g., straight Miller/Robertshaw blade) may be used. The blade portion 116 carries the one or more light sources 140. Although the one or more light sources 140 are shown on a top of the blade portion 116, in alternative embodiments, the one or more light sources 140 are disposed at one or more of the following locations: a side of the blade portion 116, a bottom of the blade portion 116, a top of the blade portion 116, a distal tip of the blade portion 116, and other locations on or relative to the blade portion 116.

With reference to FIG. 3B, in a further embodiment, in addition to or instead of the one or more light sources 140 being carried by the blade portion 116, a black light source (e.g., black light source 160) is carried by the bottom 124 of the handle 114 for emitting black light from the bottom 124 of the handle 114. In such an embodiment, a switch (e.g., switch 128) for controlling the black light source on the bottom 124 of the handle 114 is preferably located in a location other than the bottom 124 of the handle 114.

With reference to FIG. 4, an embodiment of an endotracheal tube 200 that may be used with the laryngoscope 100 (as part of an endotracheal intubation system) in performing endotracheal intubation will be described. The endotracheal tube 200 includes a clear plastic intubation tube 210 with an elongated central lumen. The intubation tube 210 includes a larger-diameter open upper end 212, which may include a cap, and a pointed open lower end or tip 214. An elongated scope (e.g., a fiberoptic scope) 220 may be received within the lumen of the intubation tube 210. The scope 220 includes a distal viewing tip 230. A plastic transparent holding sheath may cover the scope 220. A longitudinally built-in narrow malleable metal intubation stylet 240, which is a narrow piece of flat metal, may be built into the holding sheath and adhered to an outer wall of the holding sheath for controlling the geometry/shape of the scope 220/endotracheal tube 200 to match the physiology/anatomy of the patient during endotracheal intubation.

In an alternative embodiment, one or more of the intubation tube 210 and the stylet 240 include a fluorescent color or other color/substance that is enhanced by the black light or that reacts to the black light (on the entire tube 210/stylet 240 or a portion and/or component there of).

A method of performing endotracheal intubation using the laryngoscope 100 and the endotracheal tube 200 will be described. The black light source 160 of the laryngoscope 100 is activated (e.g., when the laryngoscope 100 is clicked together/assembled). The blade portion 160 of the laryngoscope 100 is inserted into the patient's mouth and behind the patient's tongue and mandible 118. By gripping the handle 114 with one's hand, the tongue and mandible 118 are lifted for viewing the vocal cords adjacent the larynx and to aid in the insertion of the endotracheal tube 200 past the vocal cords. The black light emitted from the laryngoscope 100 prompts the visible effects of fluorescence and phosphorescence with respect to the patient's vocal cords and the glottis, making the patient's vocal cords and the glottis visible either directly by the eyes of the medical provider or via the scope 220 of the endotracheal tube 200. The endotracheal tube 200 is inserted into the patient's mouth, between the patient's visible vocal cords into the larynx, and then into the trachea of the patient in a usual manner. The stylet 200 of the endotracheal tube 200 may be used to shape the scope 220/endotracheal tube 200 to the individual anatomy/pathology of the patient.

As discussed above, in the embodiment of the laryngoscope 100 shown in FIG. 1, applying pressure to the push buttons 129, 130 causes the respective light sources 150, 160 to be deactivated and removing the pressure to the push buttons 129, 130 causes the respective light sources 150, 160 to be re-activated. The medical provider operates the push buttons 129, 130 of the switch 128 (e.g., to cause only black light to be emitted, only white light to be emitted, or both types of light to be emitted) so that optimal viewing of the vocal cords occurs. The medical provider may prefer to use the black light condition and/or the white light condition, depending on external lighting conditions, the individual anatomy/pathology of the patient, the patient's condition, and other factors.

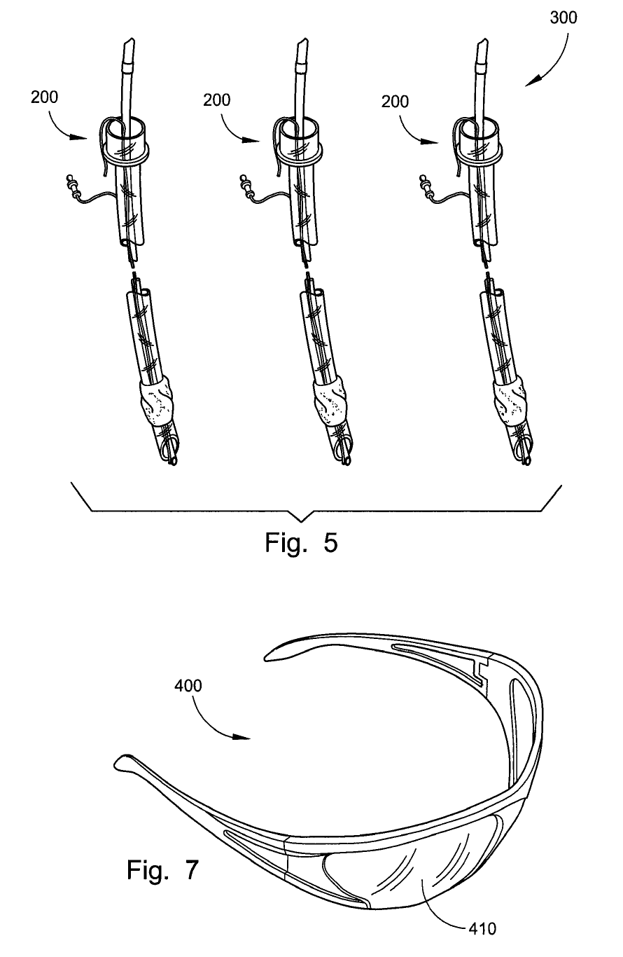

With reference to FIG. 5, in another embodiment of the above endotracheal tube 200, a kit 300 of endotracheal tubes 200, intubation tubes 210, and/or stylets 240 of different sizes (e.g., different weight, height, and/or depth; adult version, pediatric version) are color-coded with different fluorescent colors or other color/substance that is enhanced by the black light or that reacts to the black light. For example, but not by way of limitation, the intubation tube 210 of each different endotracheal tube 200 is coated with a unique fluorescent colored material or other color/substance that is enhanced by the black light or that reacts to the black light. The entire endotracheal tube 200 may include a fluorescent color or one or more portions of the endotracheal tube 200 may include one or more fluorescent colors (or other color/substance that is enhanced by the black light or that reacts to the black light). For example, but not by way of limitation, a cap of the endotracheal tube 200 may include a unique fluorescent color (or other color/substance), the intubation tube 210 may include a unique fluorescent color (or other color/substance), and/or the stylet 240 may include a unique fluorescent color (or other color/substance) for identifying the proper size endotracheal tube 200, intubation tube 210, and/or stylet 240, and for improving visualization of the endotracheal tube 200, intubation tube 210, and/or stylet 240 during endotracheal intubation. Using the black light source 160 of the laryngoscope 100, one may easily identify the correct-sized endotracheal tube 200, intubation tube 210, and/or stylet 240 by shining black light on the kit of endotracheal tubes 200, intubation tubes 210, and/or stylets 240 identified by different fluorescent colors (or other color/substance) so that the proper-sized endotracheal tube 200, intubation tube 210, and/or stylet 240 for the patient/application is selected. The different-size/fluorescent-color endotracheal tubes 200, intubation tubes 210, and/or stylets 240 are easy to identify with a black light source, especially in no-light, low-light, or dark conditions. Further, the fluorescent (or other color/substance) endotracheal tube 200, intubation tube 210, and/or stylet 240, in combination with the black light laryngoscope 100, improves visualization of the endotracheal tube 200, intubation tube 210, and/or stylet 240 passing the vocal cords during endotracheal intubation.

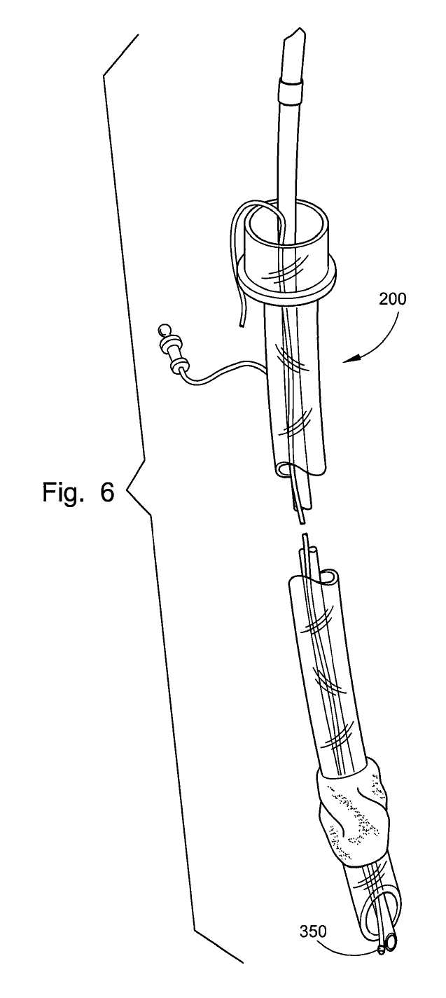

With reference to FIG. 6, in another embodiment, the stylet 240 and/or the endotracheal tube 200 includes a black light source 350 that emits black light to enhance visualization of the vocal cords during endotracheal intubation. For example, the stylet 240 and/or the endotracheal tube 200 with black light source 250 may be used in conjunction with the laryngoscope 100 discussed above with respect to FIGS. 1-3, or in conjunction with a normal laryngoscope that does not have a black light source 150.

With reference to FIG. 7, an embodiment of a pair of protection intubation glasses 400 includes lenses 410 configured (e.g., tinted to a particular shade) so that when used in conjunction with the black light sources described above during endotracheal intubation, the visibility of the vocal cords is intensified/enhanced.

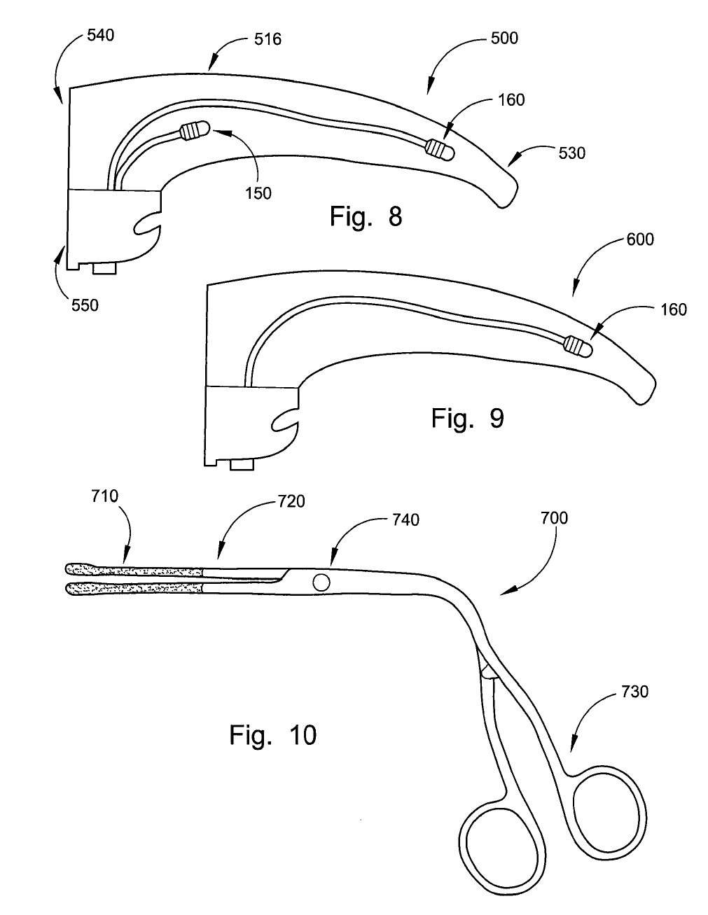

With reference to FIG. 8, an embodiment of a separate laryngoscope blade 500 that may be detachably connected to a separate laryngoscope handle (e.g., handle 114) will be described. In the embodiment shown, the blade 500 is a curved Macintosh blade; however, in alternative embodiments, other types of blades (e.g., straight Miller/Robertshaw blade) may be used.

The blade 500 may be made of stainless steel, plastic, or a combination of stainless steel and plastic. In other embodiments, other materials are used.

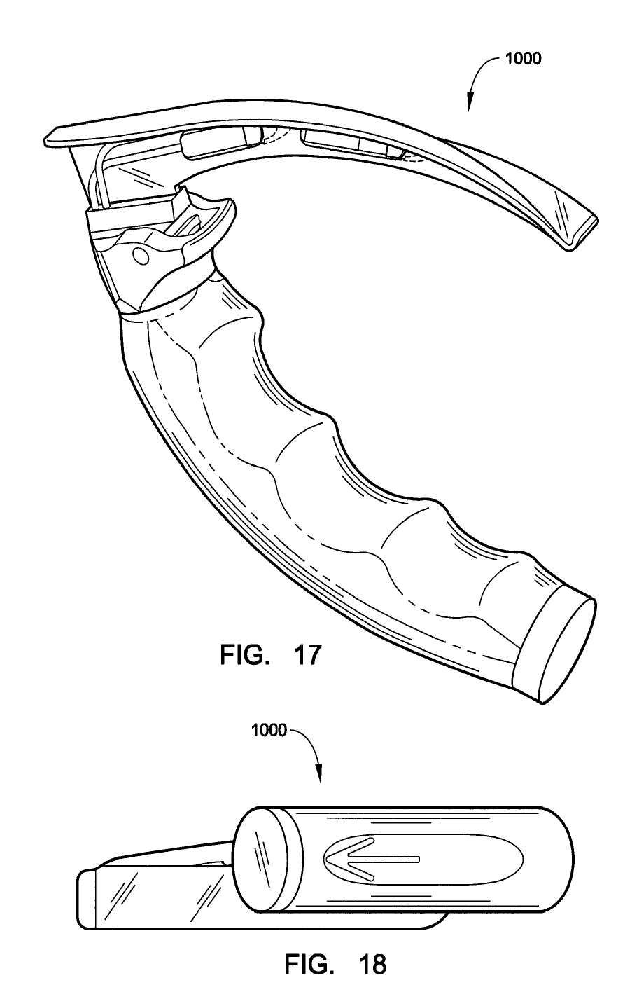

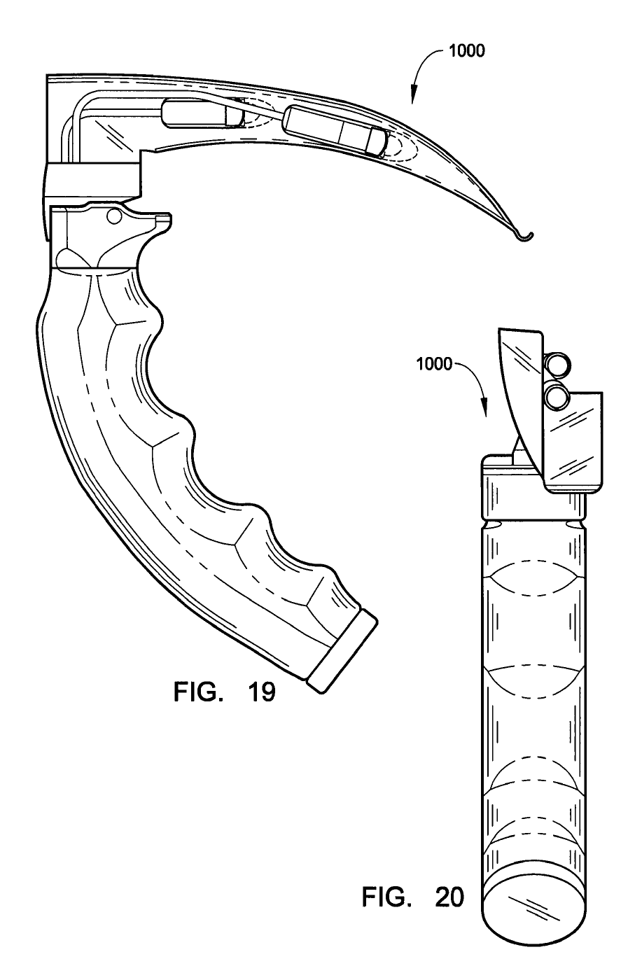

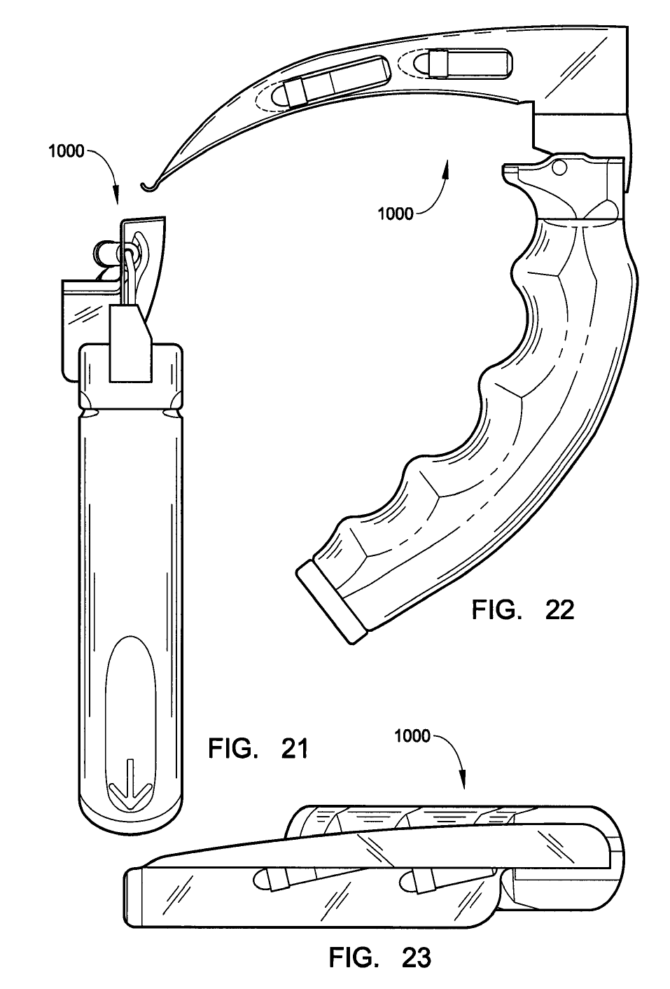

The blade 500 includes a blade portion 516 with a distal portion 530 and a proximal portion 540. The blade portion 516 carries a white light source 150 and a black light source 160. The black light source 160 is located distally of (i.e., closer to the distal portion 530 relative to) the white light source 150 on the blade portion 516. The advantage of locating the black light source 160 distally of the white light source 150 is that, in use, the proximal white light source 150 provides general illumination (e.g., of the interior of the mouth and back of the patient's throat) while the distal black light source 160, which is disposed closer to the patient's vocal cords and the glottis, provides directed black light illumination of the patient's vocal cords and the glottis, prompting the visible effects of fluorescence and phosphorescence with respect to the patient's vocal cords and the glottis. In another embodiment (FIGS. 17-23), the distal black light source 160 is disposed 1.5 to 2.0 inches in front of proximal white light source 150. In alternative embodiments, the white light source 150 and/or the black light source 160 are located at other positions/locations than those shown.

The blade 500 includes a connection section 550 for mechanically coupling the blade 500 to a top of the separate laryngoscope handle and electrically coupling the white light source 150 and the black light source 160 to a power source in the laryngoscope handle. Alternatively, the blade 500 carries its own power source for powering the white light source 150 and the black light source 160. When the blade 500 is connected to the laryngoscope handle, the connection section 550 is configured so that both the white light source 150 and the black light source 160 are automatically activated and when the blade 500 is disconnected to the laryngoscope handle, the white light source 150 and the black light source 160 are automatically deactivated. This automatic activation/deactivation may be achieved by electrical connectors and/or switch(es) (e.g., an electrically conductive ball contact on connection portion of handle and a cooperating electrically conductive ball contact on connection portion of blade 500 that contact each other to close the circuit when the blade 500 and the handle are connected and lose contact with each other to open the circuit when the blade 500 and the handle of disconnected). For example, the mechanical connection between the connection section 550 and the laryngoscope handle may put the power source in the handle in electrical communication with the white light source 150 and the black light source 160 or may cause a switch at the interface of the connection section 550 and the laryngoscope handle to be closed. In an alternative embodiment, the blade 500 and/or laryngoscope handle carries a user controllable switch to control activation/deactivation of the white light source 150 and the black light source 160 (e.g., both on, both off, one on and one off).

FIG. 9 illustrates another embodiment of a separate laryngoscope blade 600 that is the same as the blade 500, except the blade 600 includes only a black light source 160 instead of both a white light source 150 and a black light source 160.

With reference to FIG. 10, an embodiment of a pair of stainless steel disposable forceps 700 including distal portions 710 of jaws 720 coated with (e.g., dipped in) a fluorescent colored material/substance (or other color/substance) that is enhanced by the black light or that reacts to the black light is shown. In an alternative embodiment, the forceps 700 are made of a plastic material including the fluorescent colored material/substance (or other color/substance) that is enhanced by the black light or that reacts to the black light. The forceps 700 include a handle 730 at a proximal end and the distal jaws 720 at an opposite end for gripping an object. The forceps 700 pivot about a pivot section 740. In use, the forceps 700 are used to grip an object (e.g., to remove an object from the patient's body). Black light (e.g., from any of the black light devices described above and shown herein) is directed at the object and the jaws 720 of the forceps 700. The fluorescent colored material/substance is enhanced by the black light, allowing the user to see exactly where to grab the object and, most importantly, not cause damage to surrounding tissue. The user then grabs the object with the jaws 720 of the foreceps 700 and, if appropriate, removes the object from the patient's body. Although the forceps 700 in the embodiment shown are Magill-type forceps, in alternative embodiments, other types of forceps with jaws 720 coated with a fluorescent material/substance may be used.

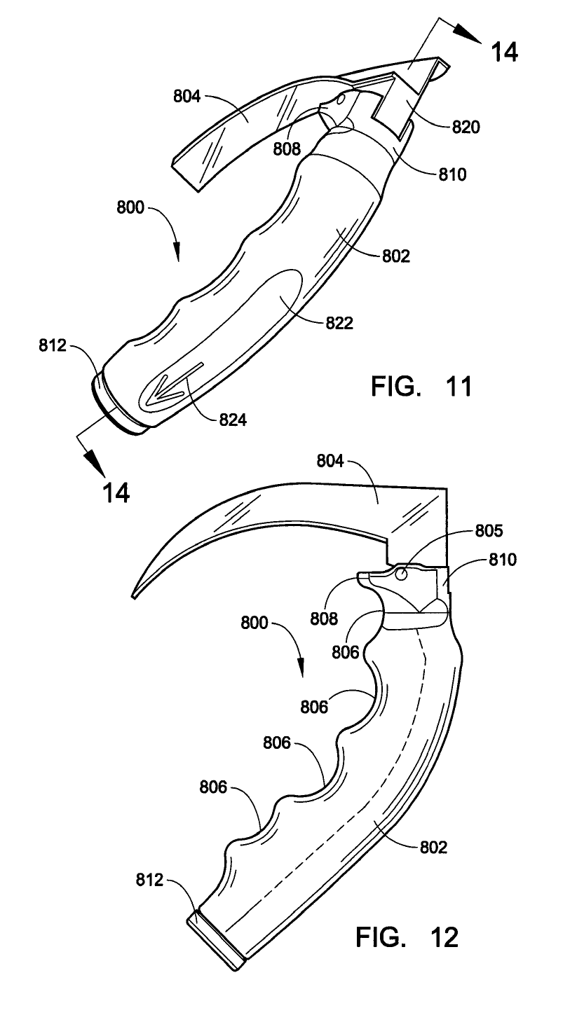

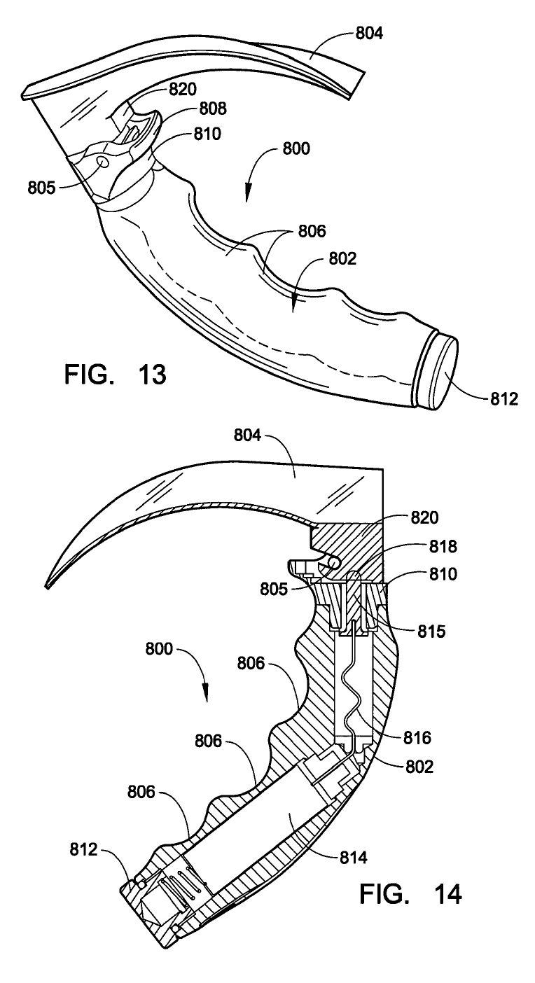

FIGS. 11 to 14 illustrate another embodiment of a laryngoscope 800 having an ergonomically shaped handle 802 and a blade 804 pivotally connected to an upper end of the handle via pivot pin 805. In one embodiment, a connection portion 820 of the blade pivots into latching engagement with a suitable conventional latching or click-lock mechanism (not illustrated) at the top of the handle when in the operative position shown in the drawings. In the embodiment shown, the blade is a curved Macintosh blade; however, in alternative embodiments, other types of blades (e.g., straight Miller/Robertshaw blade) may be used. The blade 804 carries one or more light sources (not illustrated) as illustrated and described above in connection with the embodiments of FIGS. 1 to 10, such as light sources 140 on a top surface of the blade as illustrated in FIG. 2. As described in connection with the previous embodiments, the one or more light sources may be a white light source, a black light source, or a black light source and a white light source. The one or more light sources may alternatively be disposed at one or more of the following locations: a side of the blade, a bottom of the blade, a top of the blade, a distal tip of the blade, and other locations on or relative to the blade. The light sources may be any of the alternative types of light sources as described above in connection with the preceding embodiments.

In this embodiment, handle 802 is of a continuously curved, ergonomic shape with a series of four finger grip indents 806 on its inner, concave surface. A projecting blade stop 808 is also located on the inner surface adjacent the upper end of the handle. The blade stop may be formed in an upper end cap 810 which incorporates the blade pivot mount, as illustrated in FIG. 14. In an alternative embodiment, the blade is directly connected to the top of the handle, eliminating the separate end cap 810 and replacing it with an integrally formed upper end portion of the handle of similar shape to end cap 810, including blade stop 808. The handle may be made of any suitable rigid and lightweight material such as metal or plastic.

The upper end cap 810 or end portion of the handle is pivotally assembled with the blade 804 in a similar or identical manner to the previous embodiments, and a lower end cap 812 is threadably engaged with a lower open end of the handle for access to a battery cavity containing a power source 814 (e.g. one or more dry cell batteries such as a lithium battery, single 4.5 volt battery powering a white light source and a black light source carried by the blade 804 in a manner similar to that shown in FIG. 8) which supplies power to the light source or sources, as best illustrated in FIG. 14. As illustrated in FIG. 14, the battery is connected to contact pin 815 in end cap 810 via spring loaded connector wire 816. Contact pin 814 projects into a contact recess 818 in the pivotally mounted portion 820 of the blade. Although not shown in the drawings, it will be understood that the contact in recess 818 is suitably connected to a light source or sources (not illustrated) mounted on the blade in a conventional manner, so that the light source or sources are automatically placed in communication with the power source when the blade is assembled or clicked together with the upper part of the handle in the operative condition of FIGS. 11 to 14, and are automatically turned off when the blade is released or disassembled from the upper part of the handle for storage when not in use, for example when the blade is released from the conventional click lock or latching mechanism (not illustrated) at the upper end of the handle and pivoted down towards the handle. The blade stop 808 prevents the blade 804 from contacting the handle when pivoted.

As in the previous embodiments, in alternative embodiments of the laryngoscope 800, other types of switches may be used (e.g., push-button switch, a toggle switch) on the bottom of the handle (or at other locations on the laryngoscope 800) to switch between an “off” condition and one or more of a black light condition, a white light condition, and a “both on” condition. In alternative embodiments, the switch may switch between conditions in addition to or other than an “off” condition, a black light condition, a white light condition, and a “both on” condition.

The continuously curved handle along with the finger grips 806 which are gripped by the health care provider during intubation provides an ergonomic design which is easier and more comfortable to use and which is angled more efficiently for proper actuation. As illustrated in FIGS. 11 and 12, the outer, convex surface of the handle is smoothly curved apart from an elongate recessed region 822 in which an arrow sign 824 pointing to the lower end of the handle is provided. The arrow provides an indication of the direction of use or manipulation of the handle in order to perform the procedure. The finger grips help the health care provider to hold the device securely and reduce the risk of the handle slipping out of the health care provider's hand.

The handle curvature may be a continuous curve of constant radius of curvature, or may be of varying curvature along at least part of its length. Due to the handle curvature, when the blade 804 is inserted into a patient's mouth as in FIG. 1, the lower end portion of the handle bends down rather than upwardly as is the case with straight handle 114 of FIG. 1, making it easier and more efficient for the health care provider to manipulate and position the blade while viewing the patient's trachea during the procedure. The radius of curvature of the outside surface of the handle was around 4.1 inches in one example, and may be in the range from 3 to 5 inches in some embodiments. The lower end portion of the handle may be bent or curved through an angle of around 40 to 60 degrees relative to the upper end of the handle which is attached to the blade.

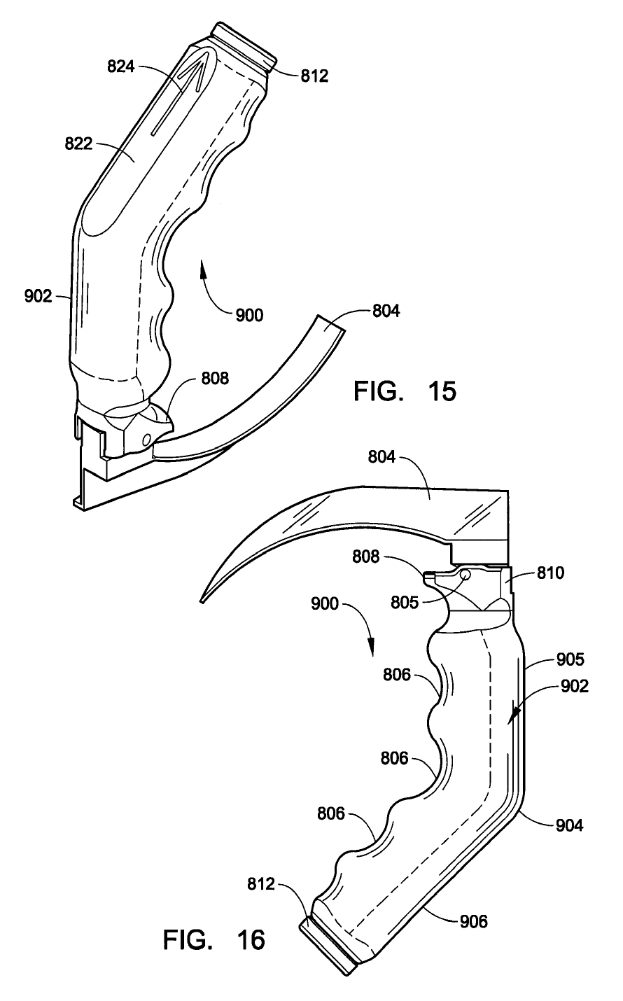

FIGS. 15 and 16 illustrate a modified laryngoscope 900 in which the handle 902 has an angled bend 904 at approximately the mid-point in its length, separating it into an upper, straight portion 905 and a lower straight portion 906 which is bent at an angle to the upper end portion. The angle between the upper portion 905 and lower portion 906 may be of the order of 40 to 60 degrees, for example 45 degrees. Apart from the handle being bent at an angle rather than continuously curved as in the embodiment of FIGS. 11 to 14, other parts of the laryngoscope of FIGS. 15 and 16 are identical to those of the previous embodiment, and like reference numerals are used for like parts as appropriate. As in the previous embodiment, the handle 902 has finger grip indents 806 and a blade stop 808 on its inner surface, facing the blade. The finger grip indents allow the health care provider to grip the handle more comfortably and securely, while the angled end portion allows the health care provider to position the blade more efficiently during the procedure.

FIGS. 17-23 illustrate another embodiment of a laryngoscope 1000 that is generally similar to the laryngoscope 800 discussion above with respect to FIGS. 11-14, which is incorporated herein, but includes a distal black light source that is disposed 1.5 to 2.0 inches in front of a proximal white light source. Portions of the distal black light source and the proximal white light source are disposed through lateral holes in a curved vertical wall of laryngoscope blade.

The above figures may depict exemplary configurations for the invention, which is done to aid in understanding the features and functionality that can be included in the invention. The invention is not restricted to the illustrated architectures or configurations, but can be implemented using a variety of alternative architectures and configurations. Additionally, although the invention is described above in terms of various exemplary embodiments and implementations, it should be understood that the various features and functionality described in one or more of the individual embodiments with which they are described, but instead can be applied, alone or in some combination, to one or more of the other embodiments of the invention, whether or not such embodiments are described and whether or not such features are presented as being a part of a described embodiment. Thus the breadth and scope of the present invention, especially in the following claims, should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; and adjectives such as “conventional,” “traditional,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the disclosure may be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases may be absent.

We claim:

1. A laryngoscope for use in viewing the vocal cords of a patient in performance of an endotracheal intubation, comprising:
a handle having an upper end, a lower end, an inner surface which faces towards a patient during use, and an outer surface;
a laryngoscope blade connected to the handle to lift the patient's tongue and mandible for viewing the vocal cords and aid in the insertion of an endotracheal tube; and
the inner surface of the handle having a plurality of spaced finger grip indents along at least part of the length of the handle between the upper and lower ends.

2. The laryngoscope of claim 1, wherein the handle has a lower end portion which is bent towards the blade.

3. The laryngoscope of claim 1, wherein the blade is pivotally connected to the handle and the inner surface of the handle has a blade stop projection extending towards the blade which restricts contact between the handle and blade when the blade is pivoted towards the handle.

4. The laryngoscope of claim 1, wherein the handle is curved along at least a major portion of its length.

5. The laryngoscope blade of claim 4, wherein the outer surface of the handle is a convex surface having a radius of curvature in the range from 3 to 5 inches.

6. The laryngoscope blade of claim 5, wherein the radius of curvature is approximately 4.1 inches.

11. The laryngoscope blade of claim 1, wherein the handle has a bend which separates the handle into an upper end portion and a lower end portion which is bent at a predetermined angle to the upper end portion.

12. The laryngoscope blade of claim 11, wherein the angle is in the range from 40 to 60 degrees.

13. The laryngoscope of claim 1, further comprising at least one of a black light source and a white light source carried by the blade.

14. The laryngoscope of claim 13, wherein the blade includes a connection section which is attached to the upper end of the handle in an operative position.

15. The laryngoscope of claim 14, wherein the connection section is configured to cause the black light source to be automatically activated when the connection section is attached to the upper end of the laryngoscope handle and automatically deactivated when the connection section is detached from the upper end of the laryngoscope handle.

16. A laryngoscope for use in viewing the vocal cords of a patient in performance of an endotracheal intubation, comprising:
a handle having an upper end, a lower end, an inner surface which faces towards a patient during use, and an outer surface;
a laryngoscope blade connected to the handle to lift the patient's tongue and mandible for viewing the vocal cords and aid in the insertion of an endotracheal tube; and
at least a lower end portion of the handle being bent inwardly towards the blade.

17. The laryngoscope of claim 16, wherein the handle is of a curved, arcuate shape along at least a major portion of its length, whereby the outer surface of the handle is convex.

18. The laryngoscope of claim 16, wherein the handle has an angled bend separating the handle into an upper portion and a lower portion bent inwardly at an angle to the upper portion.

19. The laryngoscope of claim 16, wherein the handle has a plurality of spaced finger gripping indents on the inner surface.

20. The laryngoscope of claim 16, wherein the blade is pivotally connected to the handle and the handle has a blade stop projection on its inner surface adjacent the upper end of the handle, the blade stop projection engaging the blade to restrict contact between the blade and handle when the blade is pivoted from an operative position relative to the handle.
